(19) United States
(12) Patent Application Publication
(10) Pub. No.: US 2010/0128223 A1
(43) Pub. Date: May 27, 2010

(54) METHOD, ALGORITHM AND DEVICE FOR TESTING VISUAL ACUITY

(76) Inventors: Eytan Blumenthal, Jerusalem (IL); Reuven Shamir, Jerusalem (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/522,113

(22) PCT Filed: Jan. 1, 2008
(86) PCT No.: PCT/IL08/00011

§ 371 Date: Dec. 4, 2009

Related U.S. Application Data
(60) Provisional application No. 60/878,046, filed on Jan. 3, 2007.

(51) Int. Cl. A61B 3/032

(52) U.S. Cl. 351/237; 351/246; 351/239

(57) ABSTRACT

This invention generally relates to a device and method for conducting visual acuity (VA) test and determining the size of a character presented during a VA test of a patient. The character size is selected from a predetermined allowed range of characters sizes. The method comprises steps selected inter alia from (a) obtaining minimal and maximal values; said minimal value is a size of which a character smaller than is unrecognizable for said patient; said maximal value is a size of which any character bigger than said maximal value is likely to be recognized by said patient; (b) obtaining a finite number, GAP, decreasing after each iteration so as to regulate the level of precision of the test; (c) predicting the patient's response; (d) presenting one or more characters and receiving the patient's response; (e) if the patient was correct, calculating character size in the range between the minimal value and the former character size; otherwise, calculating character size in the range between the maximal value and the form size; (f) updating said minimal and maximal values such that a new maximal value is decreased by a function of GAP and the minimal size; value is increased by a function of GAP; and, (g) terminating the test when GAP is smaller than a predetermined value and determine VA as the average between said updated minimal and maximal values.

### FIELD OF THE INVENTION

The present invention generally pertains to methods, algorithm and device for testing visual acuity.

### BACKGROUND OF THE INVENTION

Visual acuity (VA) is the single most important piece of information obtained during an eye examination. Great importance is attached to it, as well as to any change noted. Its importance is reflected by the fact that it was chosen to be the primary endpoint in numerous clinical trials on macular degeneration, cataract surgery, retinal photocoagulation, refractive surgery and others. Visual acuity is a subjective test in which the patient is tested with characters of increasingly smaller font, to determine the smallest size that can be recognized with certainty. Visual acuity quantifies the finest print that can be resolved by the macula, and is a measure of the health of the eye, as well as the adequacy of the refraction prescription (glasses) worn.

As such, VA testing should ideally benefit from the latest development in both computerized technology and diagnostic algorithms. While some may argue that the Early Treatment of Diabetic Retinopathy Study (ETDRS) chart has revolutionized and standardized VA testing, the ETDRS VA test methodology continues to rely on a manual, relatively simple, diagnostic approach, which does not incorporate a computerized thresholding algorithm.

The standard of care today, in respect to testing visual acuity, at both the ophthalmologist and optometrist clinic, is centered around a manual test in which the patient is asked to read progressively smaller characters, from a printed or projected chart, usually either the Snellen or ETDRS charts. The examiner points to progressively smaller character rows until the patient ca not longer resolve the characters presented to him. While computerized charts (characters presented on a computer monitor), as well as overhead projectors exist for presenting the Snellen or ETDRS chart, either in full or one line at a time, this test has largely remained a manual test where the patient is asked to identify characters of progressively smaller font, shown in a chart configuration. As a general rule, all patients are show the exact same chart, with only one exception, that in the computerized charts it is possible to randomize the letters shown, such that the patient cannot memorize the test shown to the right eye, when being examined in the left eye (or from one day to the next).

While it may be argued that variability and fluctuations in the subjective human response limit any potential benefits of incorporating more refined approaches, others believe that the progress made during the past 20 years of automated VF testing serves as evidence contradicting this opinion. In fact, precisely because of the variability inherent in these subjective psychophysical tests, averaging multiple responses, as well as utilizing thresholding algorithms may allow more refined endpoints.

Others may argue that more refined VA results are of little clinical benefit, since VA is primarily used as a means to refract patients, and the smallest meaningful change in a prescription (¼ spherical diopter) is roughly the equivalent of one Snellen chart line.

Following are several scenarios where our limited ability to test for VA accurately may undermine our goals:
I. The enormous deterioration that occurs when a 20/200 patient deteriorates to 20/400, a change that is barely detectible using current methods.
II. The user's inability, for the purpose of clinical trials, to obtain accurate VA scores along a continuous scale (such as 20/32 vs. 20/37), along with reliability and confidence interval values. More accurate VA data can increase the power of clinical trials, enabling a decrease in sample size, or alternatively, may shorten the duration of the study.
III. The user's inability to conveniently quantify low visual acuities (such as 20/800 vs. 20/900) and the less-than-ideal measurements of “finger counting 2-feet and hand-motion 1-meter”).

In summary, it might turn out beneficial to revisit the methods currently utilized for testing visual acuity at both the clinical and the research setting.

PCT application 20040891991 discloses an apparatus for testing visual acuity of a subject is disclosed. The apparatus includes control means (e.g. a digital computer) and project means (e.g. a computer monitor). The presentation means is controlled to present a series of symbols for attempted identification by the subject. The presented size of the symbols is selected from a substantially continuous range, allowing fine measurements of visual acuity to be obtained by varying the presented size of the symbols by small increments close to the visual acuity threshold of the subject.

PCT application 09818381 discloses a visual acuity tester based on a personal computer is provided that is capable of presenting visual acuity test characters at varying sizes on a monitor. The visual acuity tester performs anti-alias the test characters to overcome the effects of staircase distortion. Preferably, the visual acuity tester performs un-weighted area sampling antialiasing. The visual acuity tester also centers the test characters relative to the pixel array of the monitor to improve the appearance of the test characters. The test characters can be centered relative to either the center of a pixel or the corner of a pixel (a pixel coordinate). Test characters can also be centered relative to a pixel center in one dimension and a pixel coordinate in the other dimension.

U.S. Pat. No. 4,861,156 describes a visual acuity test procedure using a video capable of presenting and projecting means and a control unit. The relevant claims: the presentation of the characters in a random fashion; enables variable contrast (testing contrast sensitivity); various characters sets.

UK patent 2,397,391 discloses a visual acuity computerized test utilizing a remote control unit. The relevant claims discuss compensating for different examination distances (room lengths); various character sets.

UK patent 2,355,540 depicts VA computerized acuity test with voice recognition and with suggestions for different strategies ending the test (determining the testing endpoint).

U.S. Pat. No. 5,121,981 describes a computerized VA test with a remote control. The relevant claims discuss accommodating different length examination distances.

U.S. Pat. No. 5,880,814 discloses VA computerized test with a remote control, enables different room lengths and monitor size. The relevant claims discuss the consideration of room length compensation, monitor size compensation; centering test characters; and antialiasing.

In regard to the prior art it is understood that higher accuracy and a shorter examination time for testing VA is hence still a long felt need.

### SUMMARY OF THE INVENTION

It is one object of the invention to disclose a method of conducting visual acuity (VA) test and determining the size of a character presented during a VA test of a patient; said size is selected from a predetermined allowed range of characters sizes. The method comprises steps selected inter alia from:
a. obtaining minimal and maximal values; said minimal value is a size of which a character smaller than is unrecognizable for said patient; said maximal value is a size of which any character bigger than said maximal value is likely to be recognized by said patient;
b. obtaining a finite number, GAP, decreasing after each iteration so as to regulate the level of precision of the test;
c. predicting the patient's response;
d. presenting one or more characters and receiving the patient's response;
e. if the patient was correct, calculating character size in the range between the minimal value and the former character size; otherwise, calculating character size in the range between the maximal value and the former character size;
f. updating said minimal and maximal values such that a new maximal value is decreased by a function of GAP and the minimal value is increased by a function of GAP;
g. terminating the test when GAP is smaller than a predetermined value and determine VA as the average between said updated minimal and maximal values.

It is another object of the invention to disclose the method as defined above, additionally comprising step of at least partially determining said size of a character by former VA results of said patient.

It is another object of the invention to disclose the method as defined above, additionally comprising step of at least partially determining said size of a character by former VA results of patients belonging to the same reference group as said patient.

It is another object of the invention to disclose the method as defined above, additionally comprising step of evaluating the level of reliability of said test according to obtainable difference between a set of responses predicted by said method, and a set of responses from said patient.

It is another object of the invention to disclose the method as defined above, further comprising steps of accumulating data concerning former VA tests of said patient and/or other patients in an adaptive database, and organizing said data according to VA parameters and retrieving said data during the VA of said patient while determining the size of the next character.

It is another object of the invention to disclose the method as defined above, additionally comprising step of selecting said VA parameters from a group consisting of gender, age, date, occupation, medical history, and data concerning the reference group of said patient or any combination thereof.

It is another object of the invention to disclose the method as defined above, additionally comprising step of differing said characters in parameters selected from size, color, shape or a combination thereof.

It is another object of the invention to disclose a method of reducing the range from which the size of a character is selected during a visual acuity test (VA), said range comprising obtaining minimal and maximal values; said minimal value is a size of which a character smaller than is unrecognizable for said patient; said maximal value is a size of which any character bigger than said maximal value is likely to be recognized by said patient. The method comprises steps selected inter alia from:
a. presenting a character with a size in said range, namely between said minimal and maximal values;
b. inputting former VA's positive respond to former characters, i.e., obtaining a first value in case the patient recognizes the character, and negative respond to former characters, i.e., obtaining a second value in case the patient does not recognize the character; and, inputting sizes of said characters;
c. updating a gap defining obtainable difference between said character size and said minimal and maximal values; said gap is reduced in following iterations;
d. projecting a character in the range between said minimal and maximal values, and receiving patient's response; and,
e. increasing the value of said minimal value and decreasing the value of said maximal value, such that difference between sizes of new range and former range is determined as a function of said gap and so forth until said gap is smaller than a predetermined value.

It is another object of the invention to disclose the method as defined above, additionally comprising step of updating said gap in predetermined values.

It is another object of the invention to disclose the method as defined above, additionally comprising step of reducing the corrected range of character size by values selected from constant, inconstant or any combination thereof.

It is another object of the invention to disclose the method as defined above, additionally comprising step of updating said gap according to said patient's responses.

It is another object of the invention to disclose the methods as defined above, additionally comprising step of updating determining the size of each presented character from the entire data accumulated during the actual examination of the examined eye.

It is another object of the invention to disclose the methods as defined above, additionally comprising step of using interchangeably characters, letters, “E” letters, pictures, and other potential symbols presentations for testing visual acuity.

It is another object of the invention to disclose the methods as defined above, additionally comprising step of presenting said characters in two or more lines during at least one step of the test, such that characters of varying sizes are either gradually decreasing or increasing in size.

It is another object of the invention to disclose the methods as defined above, additionally comprising step of presenting a plurality of N characters; said N is an integer number equal or higher two; at least a portion of said N characters are either similar or different sizes.

It is another object of the invention to disclose the method as defined above, additionally comprising steps of statistically modeling the patient's responses; said model is inputted with said minimal and maximal values and with an estimated VA value; and, calculating probabilities of which said patient is capable of recognizing said character.

It is another object of the invention to disclose the method as defined above, additionally comprising step of setting the probability as 1 in case size of the presented character is bigger than said maximal value, and 0 in case said size is smaller than said minimal value.

It is another object of the invention to disclose the method as defined above, additionally comprising step of setting said probability as linear in case size of the presented character is bigger than said minimal value and smaller than said maximal value, depending on values inputted into a modeling tool.

It is another object of the invention to disclose the method as defined above, additionally comprising step of setting said probability as non-linear in case the size of the presented character is bigger than said minimal value and smaller than said maximal value.

It is another object of the invention to disclose a method of performing visual acuity (VA) examination, comprising the steps of presenting two or more characters of unequal size, such as gradually shrinking in size, and requesting the patient to identify the smallest character recognized, so as to reduce the number of steps required.

It is another object of the invention to disclose a method for performing visual acuity (VA) examination and maximizing the accuracy in said VA. The method comprises step selected inter alia from:
a. selecting at least one character from the ‘Blumenthal-Shamir font’ as described in any of FIG. 10 to FIG. 11;
b. presenting two or more characters of unequal size, such as gradually shrinking in size; and,
c. requesting the patient to identify the smallest character recognized, so as to reduce the number of steps required.

It is another object of the invention to disclose the methods as defined above, further comprising step of presenting a different number of characters at each step of the test, based on the patient's former responses.

It is another object of the invention to disclose the methods as defined above, further comprising step of simultaneously presenting two or more lines, such that said characters within each of said lines are of different sizes.

It is another object of the invention to disclose the methods as defined above, further comprising step of at least partially determining the final visual acuity score by accumulating the data during the examination, such that all responses are used by the thresholding algorithm to determine the final visual acuity.

It is another object of the invention to disclose the methods as defined above, further comprising the step of giving different balance to either correct or incorrect answers; said balance is based on the difference between the responses and the threshold along VA axis in determining VA value.

It is another object of the invention to disclose the methods as defined above, further comprising the step of presenting characters in any size, rather than pre-determined fixed sizes.

It is another object of the invention to disclose the methods as defined above, further comprising the step of eliminating a ceiling effect, such that visual acuity can be tested to the patient's real limit, limited only by the resolution of the monitor.

It is another object of the invention to disclose the methods as defined above, further comprising the step of eliminating a floor effect, by presenting large letters, limited only by the size of the monitor, thus visual acuities of “finger counting” and “hand motion” can be replaced by a quantified analysis and score.

It is another object of the invention to disclose the methods as defined above, further comprising the step of inputting a ceiling and/or floor value prior to the test, such that stimulation outside that range is not presented.

It is another object of the invention to disclose the methods as defined above, further comprising the step of inputting parameters selected from the room length, monitor size, monitor resolution, as well as additional parameters such as the font type before presenting said character such that the device will determine the size of presented letters.

It is another object of the invention to disclose the methods as defined above, further comprising step of inputting patient's former visual acuity or retrieving said data, hence utilizing said data by the testing algorithm and refining the starting point and optimizing the testing procedure.

It is another object of the invention to disclose the methods as defined above, further comprising the step of inputting patient's former reliability and variability values or retrieving said values hence utilizing by the testing algorithm to optimize the variations between succeeding presentations.

It is another object of the invention to disclose the methods as defined above, further comprising the step of using prior data for providing assumed parameter's values for the physiological model, reliability, repeatability, reproducibility, false positive and false negative.

It is another object of the invention to disclose the methods as defined above, further comprising the step of basing a starting point visual acuity score on an a priori assumption, or cumulative statistics of the patient's data or his reference group data, thus shortening the thresholding process.

It is another object of the invention to disclose the methods as defined above, further comprising the step of using former VA data of a reference group during a test sequence thus helping refine calculation of the starting point and the variations between succeeding steps and test sequence.

It is another object of the invention to disclose the methods as defined above, in which presenting the variations (changes in size) between consecutive characters to the patient take into account said statistical data, such that different VA values have different chances of being found, but a chance that is extracted from population statistics.

It is another object of the invention to disclose the methods as defined above, in which collecting a VA test provides data on reliability, repeatability, reproducibility, false positive, false negative, derived from the data accumulated collected during at least one VA tests.

It is another object of the invention to disclose the method as defined above, additionally comprising the step of determining test termination (end-point) by certain cut-off values selected from a group consisting of: reliability, repeatability, reproducibility, false positive and false negative.

It is another object of the invention to disclose the method as defined above, additionally comprising the steps of determining by the test algorithm certain test as “unreliable” and providing an “unreliability score” based on reliability, repeatability, reproducibility, false positive and false negative data or else attaches to each examination one of a group of ordinal unreliability statements.

It is another object of the invention to disclose the method as defined above, additionally comprising the step of selecting said unreliability statements are from a group consisting highly unreliable, moderately unreliable, mildly unreliable, borderline reliable, reliable and highly reliable.

It is another object of the invention to disclose the methods as defined above, further comprising the steps of repeating said VA test several times, obtaining reliability, repeatability, reproducibility, false positive and false negative data, translated into reliability parameters.

It is another object of the invention to disclose the methods as defined above, further comprising the step of presenting the number of characters presented, and/or the test duration, in an electronic record, and on the printout report.

It is another object of the invention to disclose the methods as defined above, further comprising the step of plotting the patient's responses along a multi-parameter physiological model, such as an “S” shaped frequency of seeing curve.

It is another object of the invention to disclose the methods as defined above, further comprising the step of providing the shape, width and smoothness of the obtained frequency of seeing curve data by calculating reproducibility, reliability, false positive and false negative scores.

It is another object of the invention to disclose the methods as defined above, further comprising the step of determining the center of the multi-parameter frequency of seeing curve by way of calculating the mid-point along that frequency of seeing curve.

It is another object of the invention to disclose the methods as defined above, further comprising the step of fitting the data by using a linear diagonal or other reduced computation model, instead of a more complex physiological model, thus simplifying data analysis.

It is another object of the invention to disclose the methods as defined above, further comprising the step of designing said VA test to be quicker, less accurate examination or, a longer, very accurate examination, and various options in between these two extremes.

It is another object of the invention to disclose the methods as defined above, further comprising the steps of predetermining the accuracy level vs. speed level on either an ordinal scale continuous scale; translating said pre-determined decision into the size of variations as well as by end point of the test; and, representing the level of precision of which threshold results are represented.

It is another object of the invention to disclose the methods as defined above, further comprising the steps of determining by the algorithm the test end-point, depending on repeatability and reliability of the accumulated data, false positive and false negative results, pre-defined test accuracy.

It is another object of the invention to disclose the method as defined above, additionally comprising the step of additionally defining said end-point by the examiner and/or software before the test is started, according to fixed predefined number of characters presented, or via several ordinal default settings.

It is another object of the invention to disclose the methods as defined above, further comprising the step of pre-determining the number of presentations included in a visual acuity test, such that the algorithm is terminated once the number of letters presented has reached the amount pre-set and the test length is fixed, irrespective of the patient's responses.

It is another object of the invention to disclose the methods as defined above, further comprising the step of calculating by the algorithm the estimated interim visual acuity after a predetermined amount of characters are presented, and use this value to fine-tune additional characters presented.

It is another object of the invention to disclose the methods as defined above, further comprising the step of announcing the termination (end-point) of said algorithm once one or more predetermined end-points are monitored by the algorithm.

It is another object of the invention to disclose the method as defined above, additionally comprising the step of selecting said end-points from a group consisting number of responses, time, reliability, stability, reproducibility,

It is another object of the invention to disclose the methods as defined above, further comprising the step of presenting said characters by the algorithm in a random fashion, such that the likelihood of any particular character presented is random. Hence, the test is free from expectations related to the shape and/or color and/or type of a certain character.

It is another object of the invention to disclose the methods as defined above, further comprising the steps of measuring room illumination and utilizing it to verify and report whether the illumination value was within a pre-determined acceptable range.

It is another object of the invention to disclose the methods as defined above, further comprising the steps of representing the patient's responses to the various characters during the test; storing said responses as part of the examination data; and, exporting said responses in electronic format.

It is another object of the invention to disclose the methods as defined above, further comprising the step of selecting said character from the Blumenthal-Shamir fonts and/or any group of characters of a fixed size, font, line thickness, digits or letters.

It is another object of the invention to disclose the method as defined above, additionally comprising the steps of assigning a recognition value to each character; and, reflecting the ease and/or difficulty in which it is recognized.

It is another object of the invention to disclose the method as defined above, additionally comprising the step of using said recognition values to either shrink or expand at least a portion of the characters' sizes accordingly, such that different characters assume equal recognition value or recognition value is factored into the thresholding algorithm.

It is another object of the invention to disclose the method as defined above, additionally comprising the step of calculating said recognition value for each character, and/or font, within the context of the frequency of seeing curve fit data.

It is another object of the invention to disclose the method as defined above, wherein shrinking or expanding a character size according to its recognition value score will assume a compensation factor by which that character needs to be to equal other characters, or else characters will be presented in non-compensated size, but the recognition factor will be factored into the thresholding algorithm during fitting of the data.

It is another object of the invention to disclose the method as defined above, additionally comprising the step of incorporating said recognition value will enable to use a much larger character set.

It is another object of the invention to disclose the method as defined above, additionally comprising the step of utilizing a test calibration procedure against known visual acuity standards (especially Snellen or ETDRS) and standardized characters, and by incorporating the recognition value, enabling the incorporation of different font types, a mixture of characters of different fonts, and even to mix together letters and numbers into the test.

It is another object of the invention to disclose the method as defined above, wherein the patient is either responding to each character even if not sure, or responding by null answer.

It is another object of the invention to disclose the method as defined above, additionally comprising the step of allowing an option of conducting either an examination where the patient must respond, or is capable of non-responding; said option is either predetermined, or decided during the examination, based on the patient's responses.

It is another object of the invention to disclose the method as defined above, additionally comprising the step of factoring said options into the visual acuity score, standard deviation, confidence interval, reliability, repeatability, reproducibility, false positive and/or false negative calculations.

It is another object of the invention to disclose the methods as defined above, further comprising the step of determining the variations fluctuate along the examination; said step of determining is in real-time based on the point along the examination in which the test is currently located, the patient ‘s cumulative responses and their reproducibility, consistence, false-positive, false-negative, etc.

It is another object of the invention to disclose the method as defined above, additionally comprising the step of changing the variations during the test, based upon the patient's responses.

It is another object of the invention to disclose the methods as defined above, further comprising the step of deriving the variations from a predetermined equation taking into account variables selected from a group consisting of the number of questions previously asked, the number of questions expected during the examination, either predetermined or respective to the patient's prior data, sub-population data or general data, cumulative estimations of reliability, repeatability, reproducibility, false positive and/or false negative of the individual, sub-population or population at large.

It is another object of the invention to disclose the methods as defined above, further comprising the step of increasing the accuracy of the final threshold determination by allowing the VA estimated value and the character sizes to cross the presumed threshold two or more times.

It is another object of the invention to disclose the methods as defined above, further comprising the step of widening the difference between the minimal and maximal values hence avoiding the steep portion of the physiological model, such as an S-curve.

It is another object of the invention to disclose the methods as defined above, further comprising the step of presenting the visual acuity values in either decimal scale, a fraction, or a logarithmic scale, or other spaces or scales, any population histogram, including scales not necessarily correlated with VA.

It is another object of the invention to disclose the method as defined above, additionally comprising the step of applying different scales to different portions of the visual acuity spectrum, such that a logarithmic scale might be used at low visual acuities, while a decimal scale for higher visual acuities.

It is another object of the invention to disclose the methods as defined above, further comprising the step of assuming the frequency of seeing curve to be S-shaped; or based on a different physiological model; or assume an asymmetrical configuration, hence free of symmetry assumptions when fitting the best curve to the sporadic data points reflecting the questions asked in each visual acuity test.

It is another object of the invention to disclose the methods as defined above, further comprising the step of assuming the multi-parameter physiological model, especially an S-curve, varying slopes at threshold, rather than assuming a fixed slope for calculating the S-curve or other physiological models for fitting the data.

It is another object of the invention to disclose the methods as defined above, further comprising the step of determining different characteristics of the physiological response as modeled for each patient.

It is another object of the invention to disclose the method as defined above, additionally comprising the step of providing by the characteristics of the physiological response, information ruling-in or ruling-out, certain pathological conditions, such as cataract, uncorrected refractive error, malingering, a normal response, glaucoma.

It is another object of the invention to disclose the methods as defined above, especially adapted to be used to test contrast sensitivity.

It is another object of the invention to disclose the method as defined above, additionally comprising the step of assuming in said contrast sensitivity tests one or more approaches selected from determining a predetermined sized font contrast threshold, or fonts selected from the ‘Blumenthal-Shamir fonts’, or a font being proportional to the eye's tested visual acuity, thresholding VA at a predetermined contrast, such that a visual acuity thresholding test can be performed in the range of 20% to 70% contrast.

It is another object of the invention to disclose a device useful in visual acuity test, comprising
a. at least one input means, adapted to receive the patient's response,
b. output means adapted to present the character,
c. processing means, adapted to calculate the next size of the character and to determine when to stop the examination.

It is another object of the invention to disclose the device as defined above, wherein said input means is selected from a keyboard, mouse, touch screen or any other means which require action from another person besides the patient to press any key before presenting the next character.

It is another object of the invention to disclose the device as defined above, wherein said input means is selected from a remote control or text to speech software embedded in the processing means, for converting the patient's vocal response to a binary response, correct or incorrect.

It is another object of the invention to disclose the device as defined above, wherein said output device is selected from a monitor, a module in a computer attached to an independent monitor or to a TV monitor or any other home use monitor.

It is another object of the invention to disclose the device as defined above, wherein characters can also be projected on a wall, using a projector, or else projected directly to the patient's eyes using a head-mounted or other projective equipment.

It is another object of the invention to disclose the device as defined above, wherein said processing means is either embedded in a computer using adaptive software, an independent device, or a part of a kit.

It is another object of the invention to disclose the device as defined above, further comprising a monitor.

It is another object of the invention to disclose the device as defined above, further comprises a second monitor, adapted to show data that can assist the examiner in monitoring the patient.

It is another object of the invention to disclose the device as defined above, wherein said data is selected from the S curve, and the size of the projected character, the percentage of correct responses or other.

It is another object of the invention to disclose the device as defined above, wherein used as a home device, which can be used to monitor VA towards finding the precise timing for cataract surgery.

It is another object of the invention to disclose the device as defined above, wherein incorporated in a test within an object where the test might be useful.

It is another object of the invention to disclose the device as defined above, wherein said object is selected from vehicles, guard posts, army, and factory workers, auto refractors and other refractive and/or optometric-type equipment.

It is another object of the invention to disclose the device as defined above, further comprises database adapted to store former results of VA tests, the number of presented figures, reference groups' results, estimations, or any other related parameter.

It is another object of the invention to disclose the device as defined above, wherein said database can be embedded in the main system or stored in a remote location or on the internet, and connected to the processing means by wires or wirelessly.

It is another object of the invention to disclose a representative mark useful in visual acuity (VA) examination; wherein said representative mark are selected from the Blumenthal-Shamir fonts.

### BRIEF DESCRIPTION OF THE FIGURES

In order to understand the invention and to see how it may be implemented in practice, a plurality of embodiments will now be described, by way of non-limiting example only, with reference to the accompanying drawings, in which

FIG. 1 schematically illustrates the results of a VA test, according to one embodiment of the present invention.

FIG. 2 schematically illustrates the results of VA test as performed nowadays, avoiding the advantages of the present invention.

FIG. 3 schematically illustrates a scheme describing an existing VA test procedure.

FIG. 4 schematically illustrates the method of providing VA tests as described in the present invention.

FIG. 5 schematically illustrates one embodiment of the character size determination algorithm.

FIG. 6 schematically illustrates another embodiment of the character size algorithm, which combines both statistical module and halving algorithm.

FIG. 7 schematically illustrates one embodiment of the two parameter model estimation.

FIG. 8A illustrates a graph of correct and incorrect responses from the patient, as the character size is increased, and FIG. 8B illustrates a graph of the percentage of correct responses as the character size is increased, so as to show the S-curve graph.

FIG. 9 illustrates a patient's VA results, described by the following ‘S’ curves.

FIGS. 10 and 11 illustrate the ‘Blumenthal-Shamir fonts’.

FIG. 12 illustrates the ‘Snellen’ chart.

FIG. 13 illustrates the ‘ETDRS’ chart.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a new approach enables higher accuracy and a shorter examination time for testing VA.

A list of definitions is hereby provided, to facilitate several objects of the present invention.

A reliable patient provides consistent responses, and would hence respond in an identical way if given the same stimulus, or test again and again (regardless of the level of VA found). In contrast, an unreliable patient might score differently on repetitive tests, give different answers to the same stimuli presented over again, and show a much larger scatter when his/her responses are plotted graphically. Reliability can be quantified using various parameters which we will define and write equations for. These parameters are listed and defined below.

The term ‘reliability’, as defined above, denotes how stable, consistent, predictable a person (or test response) is. This quality can be broken up into components, that each highlights a different feature of this consistency. We will create strict definitions and equations for each, such that they will be calculated from the data set collected during a VA test. Following are the definitions for these parameters: The term ‘reliability’ also refers to the total consistency of the response, the sum of the following components. The reliability of the patient can be estimated as the min distance value that is computed from the VA estimation plotted graph. Patients that are more reliable will result with lower min distance values. This is because each “unexpected response” increases the distance value, and reliable patients, by definition, should have few “unexpected responses”. The term “unexpected response”, as opposed to erroneous response, is defined as response which is opposite to the patient's expected response for that character size. For example, if the patient did not recognize a character, but happened to guess it, this would qualify as an unexpected response.

The term ‘repeatability’ refers hereinafter to the state occurring when the full test is repeated several times, the final VA scores of consecutive tests is closely clustered. Stated otherwise, a repeatable person will score identically when taking the test multiple times.

The term ‘reproducibility’ refers hereinafter to the patient is able to function at a similar level over and over again. He/she is able to reproduce his previous results with high accuracy. There is some overlap with the above term. The way they will be calculated will differ.

The term ‘consistency’ refers hereinafter to the Consistency measures whether the reliability is constant throughout the examination, or is there a period when the responses are more reliable and other periods when the responses are less reliable.

The term ‘false positive’ refers hereinafter to the situation where the patient responds correctly, when in fact the rest of the data suggests that he shouldn't have. Classically, false positive responses are guesses that happen to be true by chance alone.

The term ‘false negative’ refers hereinafter to the situation where the patient responds incorrectly, when in fact the rest of the data suggests that he should be able to recognize that character size correctly. Classically, false positive responses are mistakes, periods of inattentiveness or can even be operator errors (for example, the response was incorrectly entered into the device).

The term ‘attentiveness’ refers hereinafter to a score that reflects the subject's loss of concentration, becoming tired, “spacing out”, etc. towards the end of the test. A subject that starts reliable but towards the end of the test turns unreliable will score low on attentiveness. In contrast, a subject that is consistently unreliable (always unreliable by the same amount), would nonetheless score high on attentiveness.

The term ‘learning effect” refers hereinafter to a measure of the improvement seen initially when first learning the test. As with any complex test, after several attempts the patient becomes familiar with the test and hence may perform better. Learning how to drive a vehicle is a typical example where a long and significant “learning effect” exists for everyone.

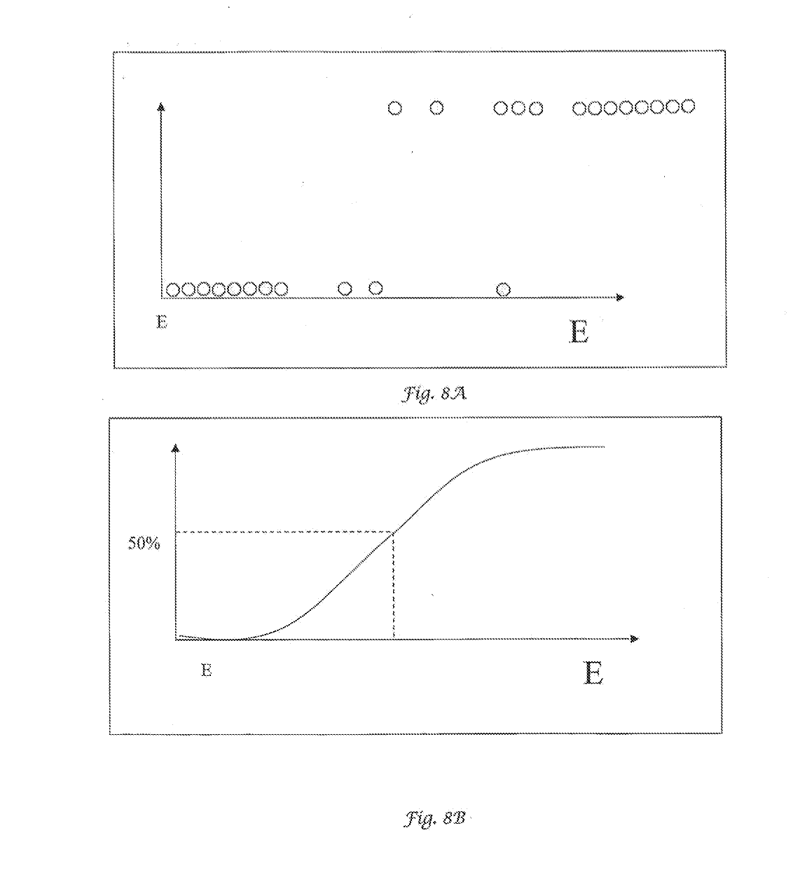

The term ‘“S”-shaped frequency of seeing curve refers to the subjective responses of an individual to a visual task, such as recognizing characters that gradually shrink in size, can be plotted on an X-Y graph. The X-axis is the size of the character, while the Y-axis is the response (Yes vs. No), as shown in FIG. 8A. Since people are not machines, there is an area of indecisiveness around the threshold, where some of the responses would be correct and some incorrect, for the same stimulus. Due to this “gray-zone” area where the responses fluctuate, the graph demonstrating the percentage of correct replies will assume an “S” shaped curve, as shown in FIG. 8B. One can perceive this physiological response as having a chance component, that increases the closer you are to the threshold zone. In fact, most would define the threshold as the point in which 50% of the stimuli are correctly identified, and 50% incorrectly. From the “S shaped” curve seen in FIG. 8B it is evident that multiple repetitions around threshold need to be made, to more accurately identify the precise value of the threshold point, which is the center of the “S shaped” curve.

The term ‘recognition value’ refers hereinafter to the ability to recognize a, particular character depends not only on its size (its font size), but also on its shape. A simplistic example is that when shown small characters of identical size, one could mistake an “8” for a “9” but would only rarely mistake a “0” for a “1”. We define this ability to recognize the character, irrespective of its size, as the “recognition value” for that character. Naturally, the recognition factor of a particular character may change based on the particular font chosen. The present invention proposes that is possible to compensate for differences in recognition value, by proportionally shrinking or enlarging each character, to make different characters as equal as possible to recognize. As an example, since the following 3 characters: “0”,“9”, “1” have very different recognition values, they will not be presented in an identical size, but instead the “0” and “1” might be slightly shrunk to make them similar to the “9” in terms of recognition (or alternatively, slightly enlarging the “9” somewhat to compensate for its lower recognition value).

The term ‘stable responses’ refers hereinafter to a stable response is one that is consistent, when the question is repeated over and over again. A person that correctly recognizes a particular character size when asked repeatedly is stable, as is a person who never recognizes a particular character size. In contrast, an unstable response implies that sometimes the person gets it right, and other times, wrong. A “reliable” patient will produce “stable responses”.

The term ‘reference group’ refers hereinafter to a group of tested individuals who's summarized data can provide information helping test a patient who is presumed to be a part of that group. For example, if high-school students are found to have good vision, the next time we face a patient who is a high-school student, we can make some a priori assumptions about his/her vision, based on known “Reference group” information.

The term ‘ceiling and/or floor effect’ refers hereinafter to unavoidable measurement errors at the very end of the measurement scale. As a simplistic example, if we measure height using a device that spans the range of 100 to 200 cm, then very short people will measure 100 cm even if they are in fact shorter, and very tall people will measure 200 cm even if they are taller than that. In VA tests where the smallest characters are 1.0 (equal to 6/6), a ceiling effect will occur such that patients with exceptional vision will score only 1.0 and not better, simply because they were not tested with characters that are smaller than the 1.0 character set.

The term ‘test calibration procedure’ refers hereinafter to the situation where differences in the surrounding settings can produce varying results. For instance, room lighting, noise, dirt on the computer monitor, the quality of the computer monitor, the length of the room, font type and numerous other factors which can influence the patient's ability to correctly identify a character shown on the computer monitor. Hence, calibration can take all these factors into account, and compensate for them in a way that when the same individual is tested in different settings, he will, nevertheless, obtain the same score. A well calibrated setting can be referred to as “standardized”.

The term ‘continuously’, in relation to font size, refers to continuity up to the limits set by the computer's precision. Most contemporary computers support 16 digits after the radix point precision (see Standard for Binary Floating-Point Arithmetic (IEEE 754), double precision 64 bit). This precision is much higher then the current VA test methods precision (one or two digits), therefore we consider it as continues scale.

The term ‘Ruling-in vs. ruling-out’ hereby explained via example: “Ruling in”: no one is fat, except those we tested and found fat (which were thus ruled-in). On the contrary, “Ruling out” refers to the case where everyone is fat, except those we tested and found slim (which were, hence, ruled-out).

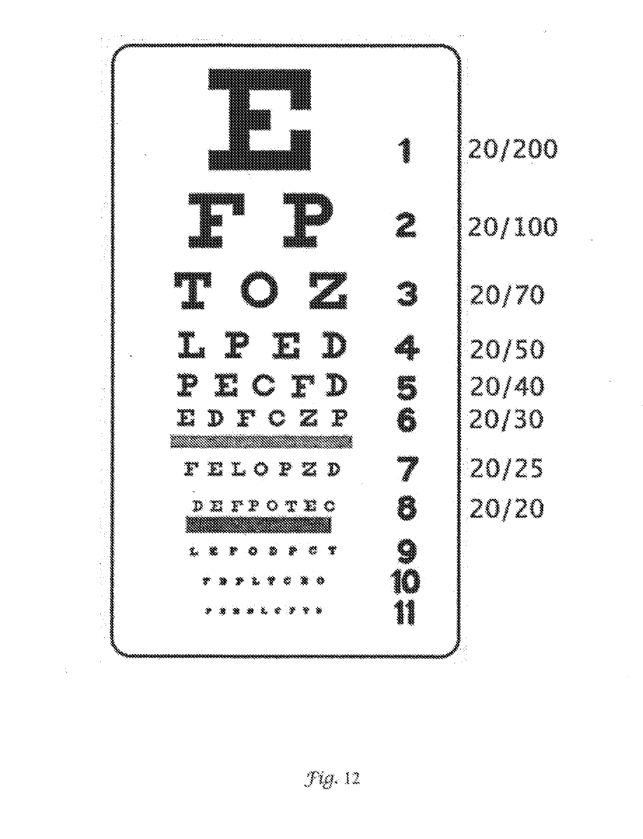

The term ‘Snellen’ refers hereinafter to a well know visual acuity standard. The ‘Snellen’ chart is depicted in FIG. 12.

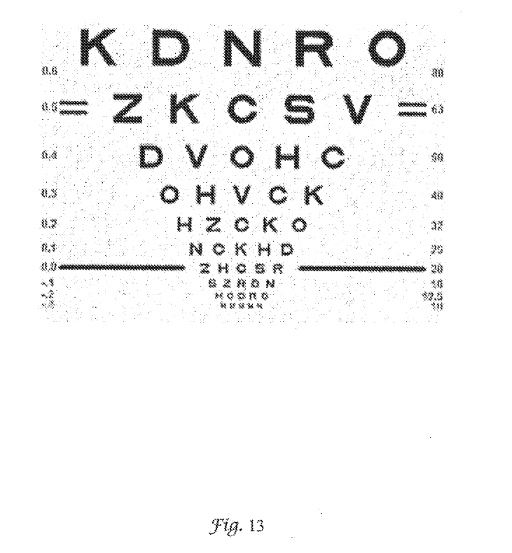

The term ‘ETDRS’ refers hereinafter to a well know visual acuity standard. The ‘ETDRS’ chart is depicted in FIG. 13.

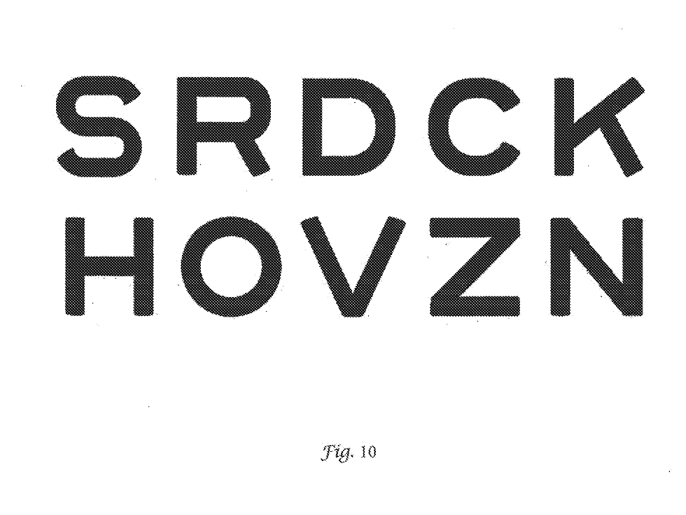

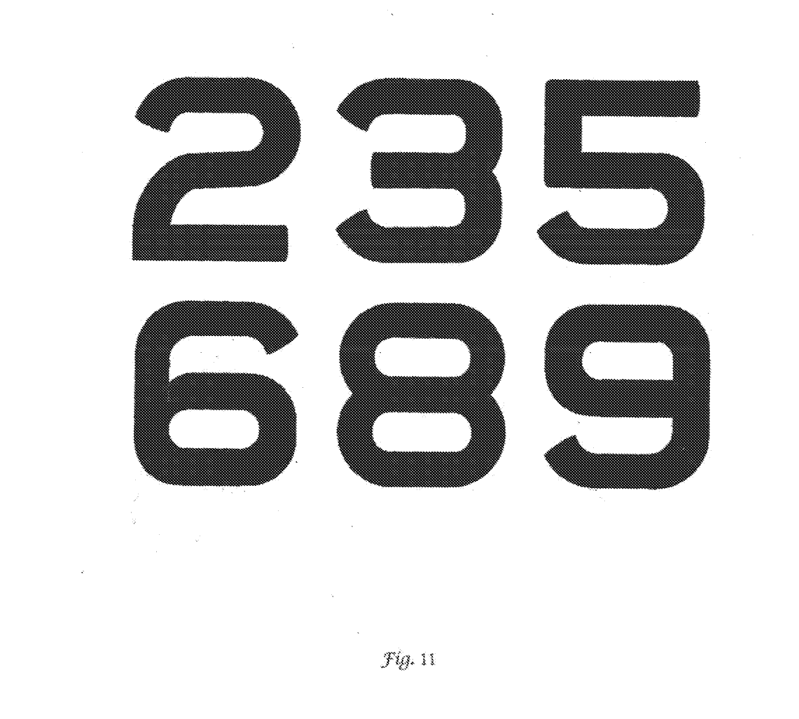

The term ‘Blumenthal-Shamir fonts’ refers hereinafter to the digit and letters fonts as depicted in FIGS. 10 and 11.

The term “visual acuities of finger counting” refers hereinafter to a well known method for visual acuities testing in which the patient is required to report the number of finger the physician is displaying.

The term “visual acuities of hand motion” refers hereinafter to a well known method for visual acuities testing in which the patient is required to report the hand movement of the physician.

It is one object of the present invention to disclose a computerized VA test hardware consists inter aliasing of a monitor, a computer and a remote control. A single character of varying sizes is presented on the monitor to the patient. The patient is asked to recognize each presented character, and the answer is inputted into the system by the examiner by any means e.g., typing it into a remote control unit. It is in the scope of the present invention wherein the input is inputted automatically, e.g., using speech to text abilities. Depending on the cumulative answers up to that point, the algorithm calculates the size of the next character to be presented. Once the test is terminated, according to the sum of the patient's responses, and utilizing a novel thresholding algorithm, VA value for this test is estimated and presented.

The present invention also depicts a computerized method of sampling, which determines the size of the next random character to be presented to the patient at each step of the test, according to at least a portion of the data formerly accumulated during the test. The aforesaid next character sizes are selected from a continuous, rather than an ordinal scale. This next character presented at each step of the test is selected randomly from a group of pre-defined characters. The sampling method may use a threshold algorithm or other methods for determining the next presented character size.

Known visual acuity summary statistics for a population can be used to refine a test sequence that is more suitable to the patient who is a known member of that population. For instance, a visual acuity test for a bus driver can benefit from data about a typical bus driver's visual acuity, thus helping refine a quicker and/or more accurate test sequence.

The present invention also depicts a threshold method that fits all the observations acquired during the test onto a mathematical, frequency-of-seeing psychophysical model, thus estimating the patient's true VA. The thresholding method uses an optimization algorithm for this mathematical fit. A reliability score for each test can be estimated using the tightness of this fit.

The present invention also depicts a database and processing module that captures, saves and analyzes the accumulating data from all tests performed. This data is later used to refine the sampling and thresholding algorithms.

It is also in the scope of the present invention wherein each eye examination is routinely started with testing the VA in each eye. It is acknowledged in this respect that such a computer-based device for testing VA is especially adapted to become a standard of care for patient encounters for both ophthalmologists and optometrists. Hence, one such device would be required in each examination lane. Testing VA in a computerized fashion coupled with a novel complex testing algorithm can provide more accurate results, as well as shortened testing time, thus reducing the burden of a manual examination from both patient and examiner. The VA test is currently performed by either: a physician (ophthalmologist), optometrist, ophthalmic technician, nurse, secretary, or other employee. A lengthy examination occupies staff time, examination lane time and patient time, all slowing clinic turnaround. The transformation of a manual diagnostic test into an electronic, device centered, test is common in medicine and includes: blood-pressure measurement, temperature measurement, weighing scale, blood-glucose testing and many other tests, whose electronic version have clearly become the standard of care. Similarly, transforming a manual visual acuity test into an electronic (computerized) one may, hopefully, become the standard of care owing to the increased accuracy, repeatability and speed.

Reliability parameters, such as reliability, reproducibility, repeatability, accuracy, false positive, false negative, 95% confidence interval (approximately −2SD→+2SD) are obtained during or after the examination and provide the system with additional aspects in regard with the patient's responses.

The abovementioned parameters are obtained using the patient's responses and the interconnections amongst. The parameters are also acquired using a group of functions such as standard deviation of repeated tests, width of the “S” curve, rate of errors outside the “S”, curve on each side (FP, FN), rate of errors outside 1 line from the VA, estimation (FP, FN), tightness of the “S” curve fit (least mean squares), symmetry of the “S” curve, number of questions to end-point, in an algorithm whose end-point is variable, and depends on estimation stability

Reliability might be related to VA, such that only after factoring VA we remain with a truer measure of reliability

It is another aspect of the present invention to present several characters with different sizes simultaneously and ask the patient to point out which is the smallest character recognized. The current dogma provides the patient with one or more characters at a time, and is asked to recognize it. Even if 5 characters are shown in a line, the patient is asked to recognize one at a time, and the examiner notes for each character whether the patient was right or wrong. In the present invention the patient is asked “which is the smallest character that you can discriminate”, such that he/she browses the line of 5 (or 2-100) characters that gradually shrink in size and notes which is the smallest one he can read. This, to us is a completely different way (conceptually) of performing the VA test, and we believe it to be entirely novel. The present invention allows the combination of the two approaches (such as: start with several rounds of asking to determine the smallest character in the line, for gross thresholding, and thereafter continue with one character per screen for fine tuning the precise threshold value).

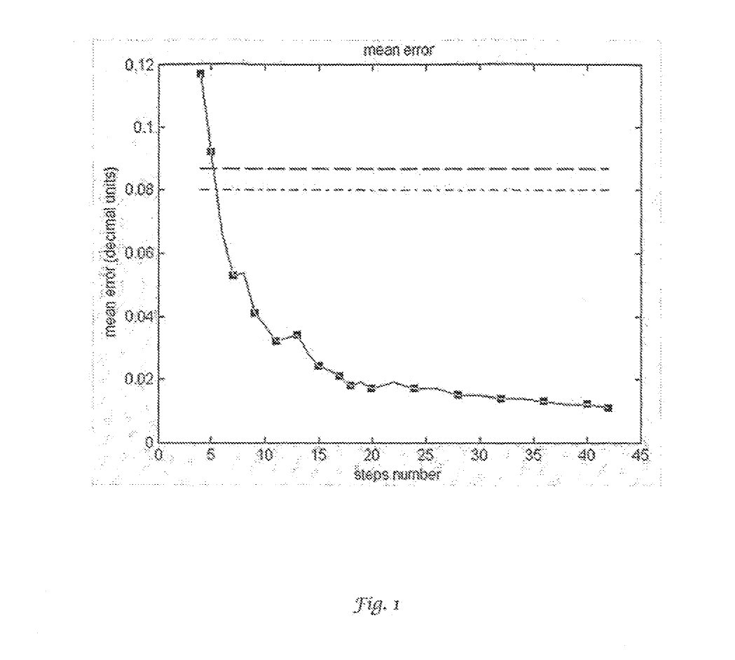

Reference is now made to FIG. 1, schematically illustrates the results of a VA test, according to one embodiment of the present invention. While the Snellen (shown in the upper line) and ETDRS (median line) performance are about 0.08 in decimal units (DU) accuracy, the suggested method is getting up to 0.01 DU.

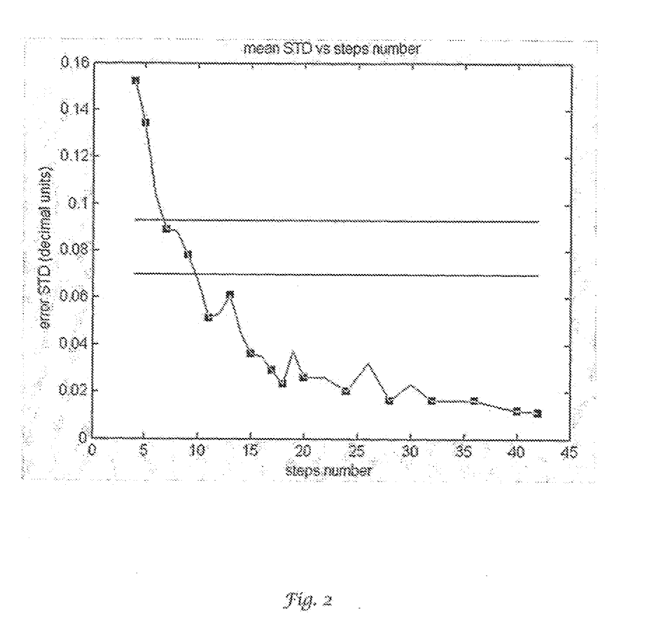

Reference is now made to FIG. 2, schematically illustrates the results of nowadays utilized VA test, avoiding the advantages of the present invention.

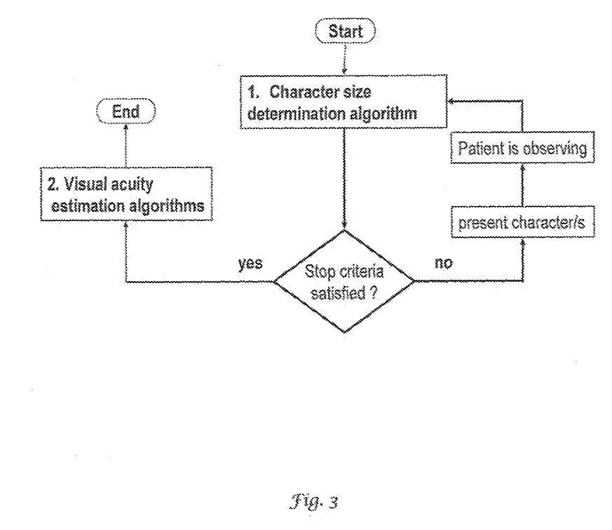

Reference is now made to FIG. 3, schematically illustrates a scheme describing an existing VA tests procedures. This prior art procedure consists of the following steps:
1. Select an arbitrary character/s size/s for the first presentation (We consider this step to be a part of the character size determination algorithm).
2. While a predefined stop criteria is not satisfied do:
   a. present characters to the patient;
   b. patient is responding to the characters;
   c. determine the next characters sizes according to the patient's response;
3. Estimate a VA value for the patient.

A common example of prior art methods is Snellen VA test chart algorithm with this scheme, comprising the following steps:
1. Start with character sizes of var_size=0.1 decimal units;
2. While the patient is recognizing most (>50%) of the characters of the last presentation
   a. patient is observing the characters;
   b. updating var_size=var_size+0.1 decimal units (this is the character size determination algorithm);
3. Estimated VA value=var_size−0.1

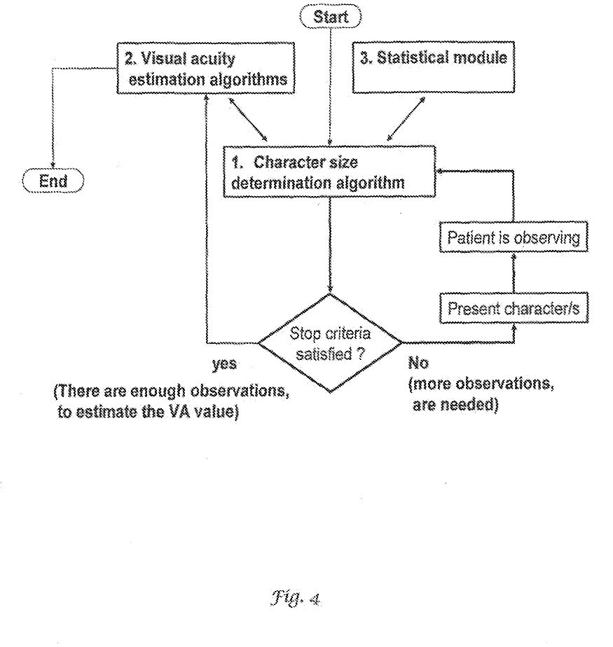

Reference is now made to FIG. 4, schematically illustrates the method of providing VA tests as described in the present invention. The major different is by incorporating the VA estimation algorithms and the statistical module. The statistical module is analyzing the past knowledge about the specific patient or/and the sub-group that the patient is belonging to or/and general population and supplying useful information to improve the VA test. The test flow is as follows:
1. Select character's size/s for the first presentation according to the statistical module information.
2. While a stop criteria is not satisfied do:
   a. Present random character's to the patient
   b. Patient is responding
   c. Determine the next characters sizes according to the patient's responses, the statistical module and the VA estimation algorithm.
3. Estimate the VA of the patient according to the accumulated data.
4. Record relevant data

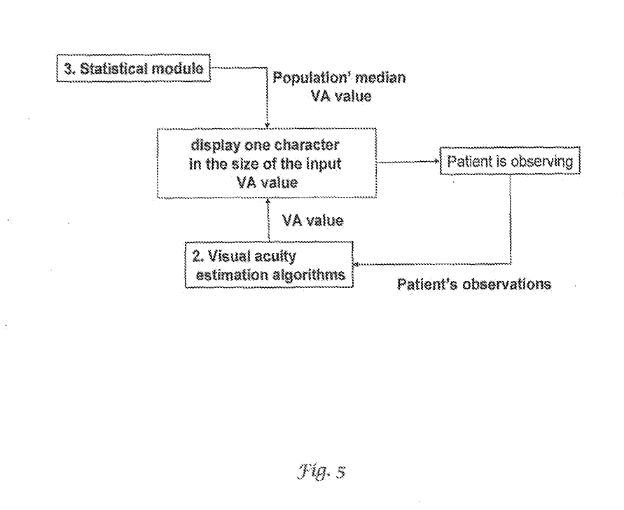

Reference is now made to FIG. 5, schematically illustrates one embodiment of the character size determination algorithm, or the algorithm to determine the size of the next presented character (this algorithm is a combination of VA estimation algorithm and the statistical module. The stopping condition was ignored for simplicity). In that embodiment, the algorithm includes the following steps:
1. The statistical module is loading the population information and computes the median VA value. current_va=the above median value. last_va=−1.
   Let us denote the required test accuracy as ACCURACY.
2. While the absolute value of the difference between current_va and last_va is bigger than the ACCURACY value, perform the following steps;
   a. present one character in the size of current_va;
   b. Patient is observing;
   c. update:
      i. last_va=current_va;
      ii. current_va=VA estimation value;
3. Estimate VA value;
4. Record relevant data;

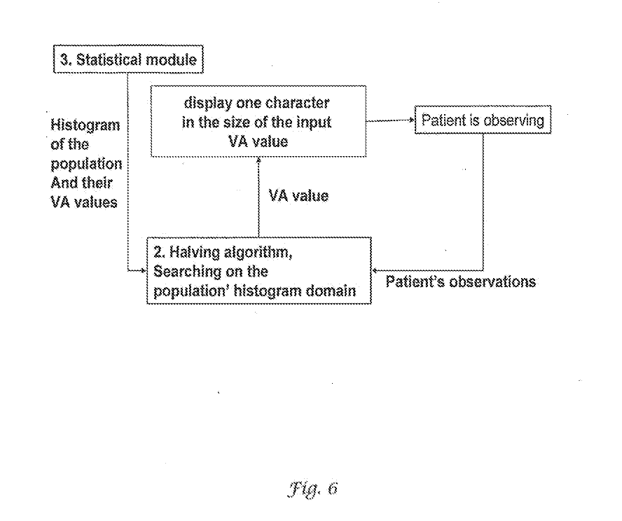

Reference is now made to FIG. 6, schematically illustrates another embodiment of the character size algorithm, which combines both statistical module and halving algorithm, such as the algorithm used in a binary search (this algorithm is a combination of having algorithm and the statistical module. The stopping condition was ignored for simplicity). The algorithm's flow is as follows:
1. The statistical module is loading the population information and computes the histogram H that counts the number of patients in each VA value range. Assume that we want to stop after five iterations. We define current_iteration=0 and adaptive histogram E, initialized as E=H.
2. While current_iteration<5
   a. present one character in the size of the median of histogram E
   b. Patient is observing
   c. current_iteration=current_iteration+1
   d. If the patient has recognized the character then E=the right half of E. otherwise, E=the left half of E
3. Estimate VA value
4. Record relevant data

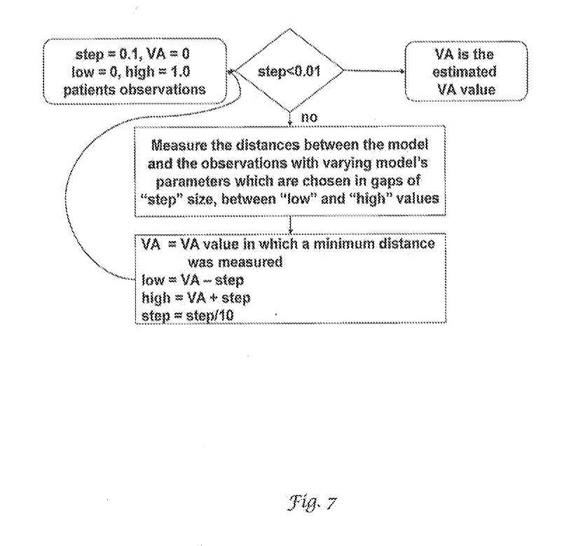

Reference is now made to FIG. 7, which is a visual acuity estimation algorithm. FIG. 7 schematically illustrates one embodiment of the two parameter model estimation. It is a combination of a multi-resolution search (optimization algorithm) and a mathematical model.

The first step is initializing the parameters' values.

The term ‘r_array’ refers to an array that is indicating if the patient had recognized the characters for each of the iterations. The value of any cell in the array r_array, for example r_array[i] equals 1 if the patient had recognized correctly the character in iteration i. Otherwise, the value of r_array[i] equals 0.

The term ‘v_array’ refers to an array with the VA values that patient was asked. The value of any cell in the array r_array, for example v_array[i] =the VA value that the patient asked to recognize in iteration i.

The term ‘N’ refers to number of observation, therefore the length of both arrays, ‘v_array’ and ‘r_array’, is equal.

The term ‘low’ refers to the lower bound of the search space

The term ‘high’ refers to the higher bound of the search space

‘min_distance’ equals a very big number

final_p1=0;

final_p2=0;

The Method

A loop runs until the parameter GAP is smaller than a predetermined value, so as to achieve a certain level of precision. The value of GAP is decreased by a constant proportion. For example, the constant is 5 and the sequence of GAP values is 1, 0.2, 0.04 and 0.008, when the predetermined value can be 0.01.

P1.x is the character size (decimal units) such that every character size with smaller decimal units (=larger character size) will be recognized correctly by the patient.

P2.x is the character size (decimal units) such that every character size with higher decimal units (=smaller character size) will be recognized incorrectly by the patient.

Let us define P1.y=1 and P2.y=0.

Model (i) is the predicted patient observation, when modeling the patient VA with values at p1.x, p2.x (the examiner searches for the p1.x and p2.x that their model is best fitting to the real patient observations)

Run Over All Gap Values
1. Run over all p1.x and p2.x values and search for the minimum distance between the model and the observations
   for p1.x=low, low+gap, low+2*gap, . . . high
   for p2.x=p1.x, p1.x+gap, p1.x+2*gap . . . high
2. Compute the distance from model to observations. The model attempts to predict the responses of the patient, and hence minimize the wrong predictions of the model.

distance=sum(∥model(p1.x, p2.x, v_array[i])−r_array[i]∥, i=1 . . . N)
3. If the computed distance is better (lower), use the p1.x and p2.x in our model.
   If (distance<min_distance){
      min_distance=distance,
      final_p1=p1
      final_p2=p2} //end if
         end of loops for p1 and p2
4. Update low and high to be near the estimated VA value

low=p1.x−gap

high=p2.x+gap

update current gap value

end of loops for p1 and p2

End of loops for gap

The method estimates the VA value as VA_value=(final_p1.x+final_p2.x)/2, and the reliability can be measured with the min distance value. Examples for mathematical models are: The first, linear model is as follows

(Assuming p1.x<p2.x, which is true by algorithm design)
{0, if p2.x<va

model (p1.x, p2.x, va)={1, if p1.x>va
p3.y where p3=p1+[(va−p1.x)/(p2.x−p1.x)][p2−p1]

The second, nonlinear model is as follows:
{0, if p2.x<va

model (p1.x, p2.x, va)={1, if p1.x>va
{else, p3.y where p3=s_curve(p1.x, p2.x, va)

Where s_curve is defined by five points on the plane (a1, a2, a3, a4, a5) defining two parabolas functions with internal dependencies. The range of s_curve values is from 0 to 1. p1.x and p2.x define the start and the end of the s_curve, and the five S curve parameters are defined accordingly. (a1, a2, a3) define the first parabola and (a3, a4, a5) define the second one. Note that using the point a3 for both of the parabolas guarantees continuity between them.

It is another object of the present invention to disclose a system adapted to implement the above described methods. The system comprises an input device, adapted to receive the patient's responses, output device, which presents the character, and processing means, adapted to calculate the next size of the character and to determine when to terminate the examination.

The input means can either be a keyboard, mouse, touch screen or any other means which require another person besides the patient to press any key before presenting the next character, or a remote control or speech to text software embedded in the processing means, for converting the patient's vocal response to a binary response, correct or incorrect. The last two means enable self examination, since the user does not need another person to input the responses. The output device can be a monitor, in a computer, attached to an independent monitor or to a TV monitor or any other home use monitor. The characters can also be projected on a wall, or by use of a projector, or projected into the eye using a head-mounted type display device.

The processing means can either be embedded in the computer, by certain software, or an independent device, or as part of a kit, with a monitor.

In another embodiment, the system comprises a second monitor, adapted to show data that can assist the examiner in monitoring the patient, for example, the S curve, and the size of the presented character, the percentage of correct responses, monitoring attention, and providing verbal feedback, or other.

The system can also be used as a home device, which can be used to periodically monitor VA, such as, for example, towards finding the precise timing for cataract surgery. For example, if the examiner set the next eye examination six months from the current examination, significant deterioration might occur within three months, implying that the date set for the next becomes too late. If, on the other hand, patients can self-monitor their VA at home, by themselves or with another person's assistance, the proper time for cataract surgery can be accurately determined. The device can either store former responses and VA values and/or any other reliability values as mentioned above, or to transmit these values to a remote database.

The remote database can be embedded in the user's computer, or on the internet. An internet web site can be used to store software which analyzes current VA results and compare it with former VA results to detect deterioration. If deterioration is detected, the system can call an alert unit in case rush operation is required.

In other embodiments, the system is incorporated in the test within other objects where the test might be useful, for example vehicles, guard posts, army, and factory workers. In some embodiments, the user cannot start operating the machinery before passing a quick VA test.

In other embodiments, the system is incorporated into other ophthalmic diagnostic or therapeutic tools to assist in VA testing, such as with auto refractors and other optometric-type equipment used for refracting patients.

The system also comprises database adapted to store former results of VA tests, the number of presented figures, reference groups' results, estimations, or any other related parameter. The database can be embedded in the main system or stored in a remote location or on the internet, and connected to the processing means by wires or wirelessly.

Reference is now made to FIGS. 10-11 which represent the ‘Blumenthal-Shamir fonts’.

The ‘Blumenthal-Shamir fonts’ comprise redesigned fonts/characters (both English alphabet characters and Roman numerals) which are a subset of characters commonly in use for testing visual acuity. The characters were designed such that the legibility of different characters is as similar as possible. In other words, the characters are as easy (or as difficult) to recognize as each other, when presented in small size. Furthermore, the characters were designed to take into account the pixalization of computer and other monitors, such that the appearance of the characters will be relatively conserved in shape and clarity even when the font size is very small. This is done by re-shaping and changing the height to width ratio from the accepted ratio found in previous VA charts.

The proportion or the ratios of the line to inter-line space in each charter and the proportion of height to width of each character were chosen to maximize the font's usefulness and accuracy in respect to testing VA.

Numeric Example

For the next character size algorithm the system uses a halving algorithm. Here, the first character size was selected arbitrary to be 0.5 decimal units (DU), and then according to the patient's observations the character size will change in gaps of: 0.3, 0.2, 0.1 and 0.05 DU. the VA test is finished after the presentation of five characters.

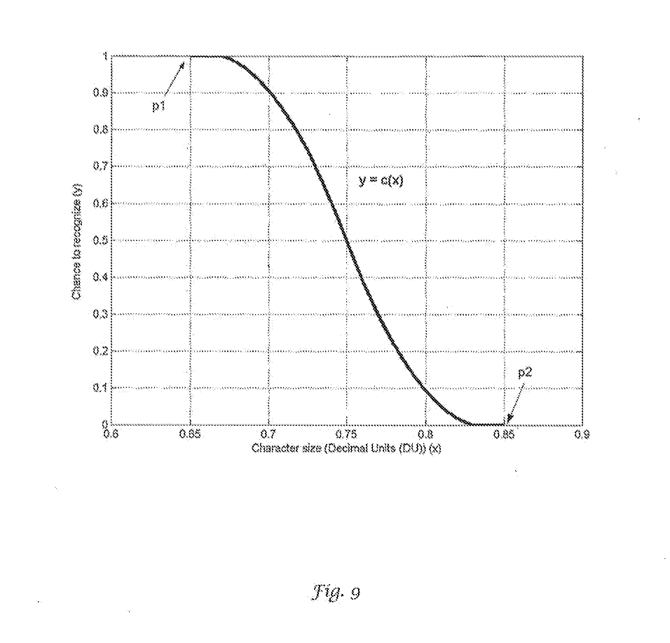

This example assumes that the patient VA can be described as in FIG. 9. The X axis shows the character size in decimal units and the Y axis shows the probability to recognize the character. It is shown that the probability to recognize the character approaches 1, when the size is about 0.65, and the probability decreases dramatically to about approach 0 when the size is about 0.85, again, in decimal units.

While assuming that this is the test flow:
1. A random character of size 0.5 DU is presented to the patient→patient recognizes the character (according to the graph)→next character size is 0.8 (=0.5+0.3) DU.
2. A random character of size 0.8 DU is presented to the patient→patient recognizes the character with chance of 10% and not recognizing the character with chance of 90% (according to the graph)→assuming that the patient did not recognize the character, the next character size is 0.6 (=0.8−0.2) DU.
3. A random character of size 0.6 DU is presented to the patient→patient recognizes the character (according to the graph)→the next character size is 0.7 (=0.6+0.1) DU.
4. A random character of size 0.7 DU is presented to the patient→patient recognizes the character with chance of 90% and not recognizing the character with chance of 10% (according to the graph)→assuming that the patient had recognized the character, the next character size is 0.75 (=0.7+0.05) DU.
5. A random character of size 0.75 DU is presented to the patient→patient recognizes the character with chance of 50% and not recognizing the character with chance of 50% (according to the graph)→let us assume that the patient did not recognize the character.

The example can summarize the test observations in two arrays:

An example of one step in the VA value estimation algorithm that presented previously: (the entire algorithm may calculate this distance for 200 times) In this case:

Input:

r_array={1, 0, 1, 1, 0}, v_array={0.5, 0.8, 0.6, 0.7, 0.75}, N=5, low=0.0, high=1.0

Initialization:

min distance=1,000,000, final_p1=0, final_p2=0;

Algorithm:

Let us denote:

model (p1.x, p2.x, v_array[i])=m(i), r_array[i]=r(i)

The linear model is used in this example.

distance=sum|(m(i)-r(i)|, i=1 . . . N)

Let us calculate the model for p1.x=0.6, p2.x=0.7, va=v_array[i]

{i=1→p1.x=0.6>0.5=va→m(1)=1, (remember that r(1)=1),

i=2→p2.x=0.7<0.8=va→m(2)=0, (remember that r(2)=0),

i=3→p1.x=0.6=0.6=va→m(3)=1, (remember that r(3)=1),

i=4→p2.x=0.7=0.7=va→m(4)=0, (remember that r(4)=1),

i=5→p2.x=0.7<0.75=va→m(5)=0, (remember that r(5)=0)}

So the distance id

distance=|(m(1)−r(1))|+|(m(2)−r(2))|+|(m(3)−r(3))|+|(m(4)−r(4))|+|(m(5)−r(5))|=|(1−1)|+|(0−0)|+|(1−1)|+|(0−1)|+|(0−0)|=1.

Now, the algorithm calculates all the distances for all the possible VA values in predefined gaps, and estimates the VA values as the mean of p1.x and p2.x correlated with the minimal distance.

1-96. (canceled)

97. A method of conducting visual acuity (VA) test and determining the size of a character presented during a VA test of a patient; said size is selected from a predetermined allowed range of characters sizes; said method comprising:
a. obtaining minimal and maximal values; said minimal value is a size of which a character smaller than is unrecognizable for said patient; said maximal value is a size of which any character bigger than said maximal value is likely to be recognized by said patient;
b. obtaining a finite number, GAP, decreasing after each iteration so as to regulate the level of precision of the test;
c. predicting the patient's response;
d. presenting one or more characters and receiving the patient's response;
e. if the patient was correct, calculating character size in the range between the minimal value and the former character size; otherwise, calculating character size in the range between the maximal value and the former character size;
f. updating said minimal and maximal values such that a new maximal value is decreased by a function of GAP and the minimal value is increased by a function of GAP;
g. terminating the test when GAP is smaller than a predetermined value and determine VA as the average between said updated minimal and maximal values
h. at least partially determining said size of a character by former VA results of said patient
i. at least partially determining said size of a character by former VA results of patients belonging to the same reference group as said patient
j. evaluating the level of reliability of said test according to obtainable difference between a set of responses predicted by said method, and a set of responses from said patient.

98. The method according to claim 97, further comprising steps of
a. accumulating data concerning former VA tests of said patient and/or other patients in an adaptive database, and organizing said data according to VA parameters and retrieving said data during the VA of said patient while determining the size of the next character and
b. selecting said VA parameters from a group consisting of gender, age, date, occupation, medical history, and data concerning the reference group of said patient or any combination thereof
c. differing said characters in parameters selected from size, color, shape or a combination thereof

99. The method according to claim 97 additionally comprising steps of reducing the range from which the size of a character is selected during a visual acuity test (VA), said range comprising obtaining minimal and maximal values; said minimal value is a size of which a character smaller than is unrecognizable for said patient; said maximal value is a size of which any character bigger than said maximal value is likely to be recognized by said patient; said method comprising:
a. presenting a character with a size in said range, namely between said minimal and maximal values;
b. inputting former VA's positive respond to former characters, i.e., obtaining a first value in case the patient recognizes the character, and negative respond to former characters, i.e., obtaining a second value in case the patient does not recognize the character; and, inputting sizes of said characters;
c. updating a gap defining obtainable difference between said character size and said minimal and maximal values; said gap is reduced in following iterations;
d. projecting a character in the range between said minimal and maximal values, and receiving patient's response; and,
e. increasing the value of said minimal value and decreasing the value of said maximal value, such that difference between sizes of new range and former range is determined as a function of said gap and so forth until said gap is smaller than a predetermined value
f. updating said gap in predetermined values
g. reducing the corrected range of character size by values selected from constant, inconstant or any combination thereof
h. updating said gap according to said patient's responses.

100. The method according to claim 97, additionally comprising steps of
a. updating determining the size of each presented character from the entire data accumulated during the actual examination of the examined eye.
b. using interchangeably characters, letters, “E” letters, pictures, and other potential symbols presentations for testing visual acuity.
c. presenting said characters in two or more lines during at least one step of the test, such that characters of varying sizes are either gradually decreasing or increasing in size
d. presenting a plurality of N characters; said N is an integer number equal or higher two; at least a portion of said N characters are either similar or different sizes
e. statistically modeling the patient's responses; said model is inputted with said minimal and maximal values and with an estimated VA value; and, calculating probabilities of which said patient is capable of recognizing said character.

101. The method according to claim 97, additionally comprising step of setting the probability as 1 in case size of the presented character is bigger than said maximal value, and 0 in case said size is smaller than said minimal value and setting said probability as linear in case size of the presented character is bigger than said minimal value and smaller than said maximal value, depending on values inputted into a modeling tool and setting said probability as non-linear in case the size of the presented character is bigger than said minimal value and smaller than said maximal value.

102. The method according to claim 97, comprising the steps of presenting two or more characters of unequal size, such as gradually shrinking in size, and requesting the patient to identify the smallest character recognized, so as to reduce the number of steps required and selecting at least one character from the ‘Blumenthal-Shamir font’ as described in any of FIG. 10 to FIG. 11;
a. presenting two or more characters of unequal size, such as gradually shrinking in size; and,
b. requesting the patient to identify the smallest character recognized, so as to reduce the number of steps required, said method further comprising steps of presenting a different number of characters at each step of the test, based on the patient's former responses and simultaneously presenting two or more lines, such that said characters within each of said lines are of different sizes,
c. determining the final visual acuity score by accumulating the data during the examination, such that all responses are used by the thresholding algorithm to determine the final visual acuity giving different balance to either correct or incorrect answers; said balance is based on the difference between the responses and the threshold along VA axis in determining VA value presenting characters in any size, rather than pre-determined fixed sizes
d. eliminating a ceiling effect, such that visual acuity can be tested to the patient's real limit, limited only by the resolution of the monitor, eliminating a floor effect, by presenting large letters, limited only by the size of the monitor, thus visual acuities of “finger counting” and “hand motion” can be replaced by a quantified analysis and score
e. inputting a ceiling and/or floor value prior to the test, such that stimulation outside that range is not presented
f. inputting parameters selected from the room length, monitor size, monitor resolution, as well as additional parameters such as the font type before presenting said character such that the device will determine the size of presented letters
g. inputting patient's former visual acuity or retrieving said data, hence utilizing said data by the testing algorithm and refining the starting point and optimizing the testing procedure
h. inputting patient's former reliability and variability values or retrieving said values hence utilizing by the testing algorithm to optimize the variations between succeeding presentations
i. using prior data for providing assumed parameter's values for the physiological model, reliability, repeatability, reproducibility, false positive and false negative.

103. The method as in claims 97 further comprising the step of basing a starting point visual acuity score on an a priori assumption, or cumulative statistics of the patient's data or his reference group data, thus shortening the thresholding process and
a. using former VA data of a reference group during a test sequence thus helping refine calculation of the starting point and the variations between succeeding steps and test sequence
b. presenting the variations (changes in size) between consecutive characters to the patient take into account said statistical data, such that different VA values have different chances of being found, but a chance that is extracted from population statistics
c. collecting a VA test provides data on reliability, repeatability, reproducibility, false positive, false negative, derived from the data accumulated collected during at least one VA tests
d. determining test termination (end-point) by certain cut-off values selected from a group consisting of: reliability, repeatability, reproducibility, false positive and false negative
e. determining by the test algorithm certain test as “unreliable” and providing an “unreliability score” based on reliability, repeatability, reproducibility, false positive and false negative data or else attaches to each examination one of a group of ordinal unreliability statements
f. selecting said unreliability statements are from a group consisting highly unreliable, moderately unreliable, mildly unreliable, borderline reliable, reliable and highly reliable
g. repeating said VA test several times, obtaining reliability, repeatability, reproducibility, false positive and false negative data, translated into reliability parameters
h. presenting the number of characters presented, and/or the test duration, in an electronic record, and on the printout report
i. plotting the patient's responses along a multi-parameter physiological model, such as an “S” shaped frequency of seeing curve
j. providing the shape, width and smoothness of the obtained frequency of seeing curve data by calculating reproducibility, reliability, false positive and false negative scores
k. determining the center of the multi-parameter frequency of seeing curve by way of calculating the mid-point along that frequency of seeing curve
l. fitting the data by using a linear diagonal or other reduced computation model, instead of a more complex physiological model, thus simplifying data analysis
m. designing said VA test to be quicker, less accurate examination or, a longer, very accurate examination, and various options in between these two extremes.

104. The method as in claim 103, further comprising the steps of predetermining the accuracy level vs. speed level on either an ordinal scale continuous scale; translating said pre-determined decision into the size of variations as well as by end point of the test; and, representing the level of precision of which threshold results are represented.

105. The method as in claim 104, further comprising the steps of determining by the algorithm the test end-point, depending on repeatability and reliability of the accumulated data, false positive and false negative results, pre-defined test accuracy and
a. defining said end-point by the examiner and/or software before the test is started, according to fixed predefined number of characters presented, or via several ordinal default settings
b. pre-determining the number of presentations included in a visual acuity test, such that the algorithm is terminated once the number of letters presented has reached the amount pre-set and the test length is fixed, irrespective of the patient's responses
c. calculating by the algorithm the estimated interim visual acuity after a predetermined amount of characters are presented, and use this value to fine-tune additional characters presented
d. announcing the termination (end-point) of said algorithm once one or more predetermined end-points are monitored by the algorithm
e. selecting said end-points from a group consisting number of responses, time, reliability, stability, reproducibility,
f. presenting said characters by the algorithm in a random fashion, such that the likelihood of any particular character presented is random and the test is free from expectations related to the shape and/or color and/or type of a certain character
g. measuring room illumination and utilizing it to verify and report whether the illumination value was within a pre-determined acceptable range
h. representing the patient's responses to the various characters during the test; storing said responses as part of the examination data; and, exporting said responses in electronic format
i. selecting said character from the Blumenthal-Shamir fonts and/or any group of characters of a fixed size, font, line thickness, digits or letters
j. assigning a recognition value to each character; and, reflecting the ease and/or difficulty in which it is recognized
k. using said recognition values to either shrink or expand at least a portion of the characters' sizes accordingly, such that different characters assume equal recognition value or recognition value is factored into the thresholding algorithm
l. calculating said recognition value for each character, and/or font, within the context of the frequency of seeing curve fit data further wherein shrinking or expanding a character size according to its recognition value score will assume a compensation factor by which that character needs to be to equal other characters, or else characters will be presented in non-compensated size, but the recognition factor will be factored into the thresholding algorithm during fitting of the data.

106. The method according to claim 105, additionally comprising the step of incorporating said recognition value will enable to use a much larger character set and
a. utilizing a test calibration procedure against known visual acuity standards (especially Snellen or ETDRS) and standardized characters, and by incorporating the recognition value, enabling the incorporation of different font types, a mixture of characters of different fonts, and even to mix together letters and numbers into the test
b. patient either responding to each character even if not sure, or responding by null answer.

107. The method according to claim 106 including
a. allowing an option of conducting either an examination where the patient must respond, or is capable of non-responding; said option is either predetermined, or decided during the examination, based on the patient's responses
b. factoring said options into the visual acuity score, standard deviation, confidence interval, reliability, repeatability, reproducibility, false positive and/or false negative calculations
c. determining the variations fluctuate along the examination; said step of determining is in real-time based on the point along the examination in which the test is currently located, the patient's cumulative responses and their reproducibility, consistence, false-positive, false-negative, etc.
d. changing the variations during the test, based upon the patient's responses.

108. The method according to claim 107 comprising steps of deriving the variations from a predetermined equation taking into account variables selected from a group consisting of the number of questions previously asked, the number of questions expected during the examination, either predetermined or respective to the patient's prior data, sub-population data or general data, cumulative estimations of reliability, repeatability, reproducibility, false positive and/or false negative of the individual, sub-population or population at large.
a. increasing the accuracy of the final threshold determination by allowing the VA estimated value and the character sizes to cross the presumed threshold two or more times and
b. widening the difference between the minimal and maximal values hence avoiding the steep portion of the physiological model, such as an S-curve.

109. The method as in claim 97, further comprising the steps of presenting the visual acuity values in either decimal scale, a fraction, or a logarithmic scale, or other spaces or scales, any population histogram, including scales not necessarily correlated with VA.
a. applying different scales to different portions of the visual acuity spectrum, such that a logarithmic scale might be used at low visual acuities, while a decimal scale for higher visual acuities
b. assuming the frequency of seeing curve to be S-shaped; or based on a different physiological model; or assume an asymmetrical configuration, hence free of symmetry assumptions when fitting the best curve to the sporadic data points reflecting the questions asked in each visual acuity test
c. assuming the multi-parameter physiological model, especially an S-curve, varying slopes at threshold, rather than assuming a fixed slope for calculating the S-curve or other physiological models for fitting the data
d. determining different characteristics of the physiological response as modeled for each patient.

110. The method according to claim 109, additionally comprising the step of providing by the characteristics of the physiological response, information ruling-in or ruling-out, certain pathological conditions, such as cataract, uncorrected refractive error, malingering, a normal response, glaucoma;
a. Adapting the method to be used to test contrast sensitivity
b. assuming in said contrast sensitivity tests one or more approaches selected from determining a predetermined sized font contrast threshold, or fonts selected from the ‘Blumenthal-Shamir fonts’, or a font being proportional to the eye's tested visual acuity, thresholding VA at a predetermined contrast, such that a visual acuity thresholding test can be performed in the range of 20% to 70% contrast.

111. A device useful in visual acuity test, comprising
a. at least one input means, adapted to receive the patient's response,
b. output means adapted to present the character,
c. processing means, adapted to calculate the next size of the character and to determine when to stop the examination
wherein said input means is selected from a keyboard, mouse, touch screen or any other means which require action from another person besides the patient to press any key before presenting the next character and is selected from a remote control or text to speech software embedded in the processing means, for converting the patient's vocal response to a binary response, correct or incorrect further wherein said output device is selected from a monitor, a module in a computer attached to an independent monitor or to a TV monitor or any other home use monitor and characters can also be projected on a wall, using a projector, or else projected directly to the patient's eyes using a head-mounted or other projective equipment.

112. The device according to claim 111, wherein said processing means is either embedded in a computer using adaptive software, an independent device, or a part of a kit further comprising one monitor or a second monitor, adapted to show data that can assist the examiner in monitoring the patient

113. The device according to claim 112, wherein said data is selected from the S curve, and the size of the projected character, the percentage of correct responses or other. Further wherein said device can be used as a home device, which can be used to monitor VA towards finding the precise timing for cataract surgery.

114. The device according to claim 111, wherein said device is incorporated in a test within an object where the test might be useful further wherein said object is selected from vehicles, guard posts, army, and factory workers, auto refractors and other refractive and/or optometric-type equipment

115. The device according to claim 114 further wherein said device comprises database adapted to store former results of VA tests, the number of presented figures, reference groups' results, estimations, or any other related parameter wherein said database can be embedded in the main system or stored in a remote location or on the internet, and connected to the processing means by wires or wirelessly.

116. A representative mark useful in visual acuity (VA) examination; wherein said representative mark is selected from the Blumenthal-Shamir fonts.
